(19) United States
(12) Patent Application Publication
(10) Pub. No.: US 2006/0281186 A1
(43) Pub. Date: Dec. 14, 2006

(54) Indicator composition

(75) Inventors: Hiroshi Inoue, Osaka-Shi (JP); Yamaguchi Norihiro, Osaka-Shi (JP)

Correspondence Address: HAMRE, SCHUMANN, MUELLER & LARSON, P.C., P.O. BOX 2902, MINNEAPOLIS, MN, 55402-0902, US

(73) Assignee: Sakura Color Products Corporation, Osaka-shi (JP)

(21) Appl. No.: 10/557,762

(22) PCT Filed: May 18, 2004
(86) PCT No.: PCT/JP04/07043

§ 371 Date: Nov. 18, 2005

(30) Foreign Application Priority Data
May 20, 2003 (JP) 2003-142520

(51) Int. Cl. G01N 33/00

(52) U.S. Cl. 436/130

(57) ABSTRACT

An indicator composition of the present invention comprises a gel composition which comprises a reagent comprising Schiff's reagent in which pararosaniline derivative is a coloration component, acidic substance, and water, wherein said Schiff's reagent comprises pararosaniline derivative as a coloration component and comprises sulfurous acid or metal salts thereof as a decoloration component, wherein said pararosaniline derivative is pararosaniline hydrochloride salt or rosaniline hydrochloride salt, wherein said acidic substance is an organic acid having sulfo group selected from the group of sulfosalicylic acid, p-toluenesulfonate, sulfamic acid, and metal salts and pH of the composition is adjusted to not greater than 3, and wherein pararosaniline derivative is decolored by said sulphurous acid or salts thereof and is colored under the atmosphere of formaldehyde.

### FIELD OF THE INVENTION

The present invention relates to an indicator composition. In more detail, the present invention relates to a gel indicator composition capable of detecting a detection material to be detected such as formaldehyde and the like by color changes.

### BACKGROUND OF THE INVENTION

Conventionally, as an indicator of this kind, for example, a diffusion type gas monitor has been provided in which a solid indicator composition is arranged in a concentration detecting layer (monitoring layer) through which, for example, formaldehyde gas passes, thereby detecting concentration thereof. As this kind of thing, a dried indicator of a printing material can be exemplified which uses a color reagent (U.S. Pat. No. 3,945,798). Further, a liquid indicator composition has also been provided which detects concentration of formaldehyde gas or liquid in which said gas is dissolved by a composition which includes rubeanic acid and a metal cyanide complex (U.S. Pat. No. 4,666,859).

However, in said indicator of a -dried printing material using a color reagent, since a concentration detecting layer (monitoring layer) is a solid and this indicator pertains to a gas-solid reaction in which formaldehyde gas is made to contact with this detecting layer, color reaction is slow and for example, it takes 8 to 24 hours to complete the color change. On the other hand, however, as shown in said US patent, in an indicator in which coloring is conducted by gas-liquid reaction or liquid-liquid reaction utilizing a liquid indicator composition, although coloring is very quickly made, in view of handling, said indicator coloring by gas-solid reaction can be said to be more preferable.

The object of the present invention is to provide an indicator composition with easy handling and capable of detecting a detection material to be detected such as formaldehyde and the like in a certain atmosphere in a short time.

Another object of the present invention is, in addition to the above mentioned object, to provide an indicator composition capable of detecting a detection material to be detected such as formaldehyde and the like by the gas thereof or liquid which include said gas.

The other object of the present invention is, in addition to the above mentioned objects, to provide an indicator composition capable of detecting a detection material to be detected such as formaldehyde with stable and preferable sensitivity.

### SUMMARY OF THE INVENTION

The present invention relates to a gel composition and it relates to an indicator composition which comprises a color reagent of a detection material to be detected. As specific embodiments, it relates to an indicator composition comprising at least a color reagent of a detection material to be detected, water, and a gelling agent.

As said gelling agent, such a gelling agent is preferable which can gelatinize water and can impart fluidity to a composition of the present invention.

Therefore, when this is filled and stored in closely packed containers such as films, tubes, stamps, writing instruments, and the like and at the time of usage in a detection atmosphere, by excavating holes in films, squeezing out of tubes, stamping, writing or the like, said composition can be exposed to said atmosphere, thereby capable of detecting concentration of a detection material to be detected such as formaldehyde and the like immediately. In particular, since an indicator with said composition sealed in a transparent bag-like synthetic resin film (including a laminated film) with gas barrier property such as ethylene vinyl acetate resin and the like is not liquid, it does not spill even when holes are excavated and can detect concentration in a short time and further, since color changes can be observed from outside, it can be easily utilized. When directly exposed to atmosphere which is a detection material to be detected without observation from outside, an indicator with said composition sealed in a laminated film such as an aluminum foil and the like can be used.

Thus, since the composition of the present invention is a gel indicator composition, it is easy to handle and a detection material to be detected can be detected depending on color change degrees in a short time. In addition, since it is a gel indicator composition, a detection material to be detected can be detected by both the gas and liquid which includes said gas. For example, when taking formaldehyde as a detection material to be detected, when there is not less than 0.05 ppm of formaldehyde gas present, or when there is concentration of not less than 0.25 mg/l of liquid which includes formaldehyde present, colors clearly change and the presence thereof can be detected, therefore, a detection material to be detected even with low concentration can be detected.

In addition, in the case of an indicator composition composed of a gel composition comprising a color reagent with pararosaniline derivative as a coloration component, by preferably including organic acid having sulfo group and/or by preferably preparing a composition in an acidic region in which hydrogen ion concentration is not greater than 3, an indicator composition can be prepared which can detect formaldehyde with stable and preferable sensitivity.

### DETAILED DESCRIPTION OF THE EMBODIMENTS

(Color Reagent)

A color reagent used in the present invention is not specifically limited as long as it is a reagent (including a composition) which includes a coloration component such as a coloring matter which develops colors with a detection material to be detected. Therefore, by using each of color reagent, a composition of the present invention can be applicable to such a detection material to be detected as formaldehyde, ozone, sulfur oxide SOₓ, nitrogen oxide NOₓ, and the like. These reagents are described in, for example, Environmental measurement and chemical analysis method notes: Salzmann's method, Japan unexamined patent publication Hei 9-235498, and “Dyes and chemicals” Vol 27, No.1 (1982).

(Formaldehyde)

For example, when a detection material to be detected is formaldehyde, other than pararosaniline derivative such as pararosaniline, Rosaniline, and the like which are used as coloration components of Schiff's reagent, publicly known coloration components such as Congo red, Naphthalene black, and the like are used. However, in an aqueous gel composition of the present invention, color reagents including at least one or more coloration components selected from the group of pararosaniline, rosaniline, 4-amino-3-hydrazino-5-mercapto-1,2,4-triazole, and Congo red can preferably be used. Publicly known color reagents (coloring compositions) described in U.S. Pat. No. 3,945,798 can also be used. For example, it is a coloring composition which includes pararosaniline, sodium sulfite (Na₂SO₃) and calcium chloride (CaCl₂). Also, publicly known color reagents described in U.S. Pat. No. 4,928,569 can also be used. For example, it is a coloration component which includes already mentioned Congo red, Naphthalene black, or Benzo purpurin.

In addition, when Schiff's reagent is used as a color reagent, as said pararosaniline derivative, other than para rosaniline and rosaniline which were already mentioned, para rosaniline hydrochloride, rosaniline hydrochloride, and the like can be exemplified.

Said color reagent is preferably contained in 0.005 to 5.0% by weight with respect to the total amount of the indicator composition of the present invention in coloration component amount of coloring matters and the like.

In addition, when pararosaniline derivative is used as a coloration component of Schiff's reagent, pararosaniline derivative is preferably contained in 0.005 to 2.0% by weight, and more preferably contained in 0.1 to 0.5% by weight. The above mentioned coloration component amount which exceeds 2.0% by weight brings a possibility that it is difficult to dissolve the coloration component in water and when the above mentioned coloration component amount is less than 0.005% by weight, tinting strength deteriorates in a gel composition.

As a component which decolors pararosaniline derivative as Schiff's reagent (a sulfite reagent), sulfurous acid or sulfite salt can be used. To be specific, sodium disulfite, sodium sulfite, sodium hydrogensulfite, potassium disulfite, potassium sulfite can be exemplified, however, they are not specifically limited to these. Said sulfurous acid or sulfite salt is contained in 0.005 to 5% by weight and preferably contained in 0.05 to 2% by weight with respect to the total amount of the indicator composition of the present invention. When said sulfurous acid or sulfite salt is contained in less than 0.005% by weight, color change is hard to occur in a gel composition. When said sulfurous acid or sulfite salt is contained in more than 5% by weight, detection sensitivity lowers in a gel composition.

In addition, in the case of formaldehyde gas, with a presence of not less than 0.05 ppm of formaldehyde, and in the case of liquid which includes formaldehyde, with a presence of not less than 0.25 mg/l of formaldehyde, color clearly changes thereby detecting the presence thereof.

(Sulfur Oxide: SO₂)

When a detection material to be detected is sulfur oxide, pararosaniline, rosaniline, pararosaniline derivative such as pararosaniline hydrochloride, rosaniline hydrochloride, and the like can be used likewise. In particular, pararosaniline hydrochloride is preferable. In addition, when a detection material to be detected is sulfuroxide, formalin (formaldehyde) is used together with the para rosaniline derivative. The present color reagent corresponds to replacement of a sulfite reagent by formaldehyde of an already mentioned formaldehyde indicator portion.

By including formalin (formaldehyde) together with para rosaniline derivative in a gel composition, when contacted with atmosphere of sulfur oxide, or SO₂, color changes thereby detecting sulfur oxide, or SO₂.

When a detection material-to be detected is sulfur oxide, although inclusion of 0.005 to 2.0% by weight of said pararosaniline derivative is feasible, preferable range thereof is 0.1 to 0.5% by weight. When said pararosaniline is contained in less than 0.005% by weight with respect to the total amount of the indicator composition of the present invention, tinting strength is inferior and the component amount of pararosaniline which exceeds 2.0% by weight brings a possibility that it is difficult to dissolve pararosaniline in a gel composition.

In addition, said formalin (formaldehyde) included together with pararosaniline derivative is preferably contained in 0.001 to 1.0% by weight with respect to the total amount of the indicator composition of the present invention. The preferably detectable concentration of sulfur oxide is not less than 0.05 ppm.

(Nitrogen Oxide: NO, NO₂)

When a detection material to be detected is nitrogen oxide NO or NO₂, anthraquinone dye as color reagent 1, Salzmann's reagent as color reagent 2 which reacts with nitrogen oxide to change color can be used. As anthraquinone dye, for example, anthraquinone acid dye, and direct dye can be used.

Although inclusion of 0.005 to 2.0% by weight of said anthraquinone dye is feasible, preferable range thereof is 0.1 to 0.5% by weight. When said pararosaniline derivative is contained in less than 0.005% by weight with respect to the total amount of the indicator composition of the present invention, tinting strength is inferior. Inclusion of 0.1 to 90% by weight of said Salzmann's reagent is feasible with respect to the total amount of the indicator composition of the present invention.

The preferably detectable concentration of nitrogen oxide is not less than 0.01 ppm.

(Ozone)

When a detection material to be detected is ozone, as a color reagent, dye can be used. For example, as a coloring matter which changes color by ozone, water soluble anthraquinone dye, azo dye, methine dye, diphenyl methane dye, triphenyl methane dye, and indigo dye can be used. Although inclusion of 0.005 to 2.0% by weight of said dye is feasible, preferable range thereof is 0.1 to 0.5% by weight with respect to the total amount of the indicator composition of the present invention. When said dye is contained in less than 0.005% by weight with respect to the total amount of the indicator composition of the present invention, tinting strength is inferior and the dye component amount which exceeds 2.0% by weight brings a possibility that it is difficult to dissolve the dye in a gel composition. The preferably detectable concentration of ozone is not less than 0.01 ppm.

(Gelling Agent)

It is important that a gelling agent used in the present invention is a gelling agent capable of gelatinizing a composition without affecting color reaction of a color reagent to detection material to be detected. In addition, a gelling agent is not limited as long as it is a gelling agent capable of gelatinizing water or each compounding component comprising water and color reagents. Further, it is important that in the indicator composition of the present invention, a gelling agent imparting fluidity or viscoelasticity to said composition is used. To cite examples, microbial polysaccharides and derivatives there of can be used. For example, pullulan, xanthan gum, wellan gum, rhamsan gum, succinoglycan, dextran, Alcaseagum (registered trademark, alcagum (common name)) and the like can be exemplified. Moreover, water soluble plant polysaccharide and derivative thereof can be used. For example, taraganth gum, guar gum, tara gum, locust bean gum, gati gum, arabinogalactan gum, Arabic gum, quince seed gum, pectin, starch, psyllium seed gum, pectin, carageenan, arginic acid, agar, and the like can be exemplified.

As gelling agents used in the present invention, thixotropic agents capable of imparting thixotropic property to water are preferable. As gelling agents satisfying such requirement, already mentioned gelling agents are preferable, however, particularly, succinoglycan, wellan gum, xanthan gum, Alcaseagum(registered trademark, alcagum (common name)), and guar gum can be exemplified. These can achieve precise concentration detection of formaldehyde gas and liquid including said gas without inhibiting color reaction of color reagents including at least one or more coloration components selected from the group of, for example, pararosaniline derivatives, 4-amino-3-hydrazino-5-mercapto-1,2,4-triazole, and Congo red. In addition, precise concentration detection to gases which include ozone, sulfur oxide SOₓ, and nitrogen oxide NOₓ, and to liquid which include said gases can be achieved as well.

It is preferable that said gelling agent is contained in 0.1 to 5.0% by weight with respect to the total amount of the composition of the present invention.

When a gelling agent imparting fluidity to said composition is used, it is preferable to prepare a gel indicator whose viscosity is not less than 1000 mPa·s measured by an E type viscometer (E viscometer) at a temperature of 20° C.

In addition, when a gelling agent imparting viscoelasticity to said composition such as agar is used, a gelling agent whose jelly strength is 30 to 2000 g/cm² is preferable.

Here, jelly strength of the composition of the present invention is a specified value measured by a measuring method (NIKKANSUISHIKI SOKUTEIHOU) and “jelly strength” is defined as a maximum weight (g) which withstands for 20 seconds per cm² of an agar gel surface.

(Acidic Substance)

Acidic substance can be included in the composition of the present invention. In particular, when the indicator composition comprises a gel composition used for a detection material to be detected such as formaldehyde, sulfur oxide or SO₂, and the like, in which pararosaniline is a coloration component and in which water is included, it is preferable to include acidic substance. In addition, in order to secure detection sensitivity and stability as an indicator composition, hydrogen-ion concentration or pH is set to not greater than 3, preferably not greater than 2, and further preferably not greater than 1.5. When hydrogen-ion concentration or pH exceeds 3, in the case of a gel indicator composition, when moisture vaporizes, coloring is likely to occur, thereby lowering sensitivity and stability as an indicator.

Further, in the case of said indicator composition which includes said Schiff's reagent, as the above mentioned acidic substance, it is preferable to include organic acids having sulfo group or metal salts thereof in view of improved detection sensitivity and stability as an indicator composition. To cite examples of organic acids having sulfo group or metal salts thereof, sulfosalicylic acid, p-toluenesulfonic acid, sulfamic acid, and each metal salts thereof. Among them, sulfosalicylic acid, p-toluenesulfonic acid, or each salt thereof are preferably used.

In addition, as already mentioned, said acidic substance is preferably contained in the range capable of adjusting pH to not greater than 3. Therefore, said acidic substance can be added in excess followed by adjusting pH to not greater than 3 by alkaline substance. In addition, when formaldehyde is used for a detection material to be detected, said acidic substances can be used together with organic acid having sulfo group or/and metal salts thereof and inorganic acid, and organic acid as well. In the case of a gel composition in which sulfur oxide or SO₂ is used as a detection material to be detected, inorganic acid and organic acid can be used without limitation. In addition, a body filler pigment showing acidity can be used as well.

(Other Components)

It is preferable that water is contained in at least 50% by weight with respect to the total amount of the composition of the present invention. As for others, each kind of additives can be compounded, for example, a water-soluble organic solvent such as ethylene glycol, propylene glycol, glycerin and the like, an antioxidant such as ascorbic acid, and the like, and an antiseptic mildew proofing agent. In particular, said water-soluble organic solvent is preferable in view of improved precision of concentration detection since said solvent can retain a certain hydroscopic state in a gel composition of the present invention, thereby capable of securing a certain color reaction. In addition, a water-soluble organic solvent such as said alcohol, polyhydric alcohol, and the like can be used as a wetting agent and is preferable when used as an indicator ink composition. Further, a water-soluble organic solvent for dissolving dye can also be used.

Although said water-soluble organic solvent need not be included in a composition of the present invention, it is preferable that said water-soluble organic solvent is contained in 0.1 to 20% by weight with respect to the total amount of the composition of the present invention, and more preferably contained in 1 to 10% by weight. When said water-soluble organic solvent is included in more than 20% by weight with respect to the total amount of the composition of the present invention, precision (sensitivity) of concentration detection lowers.

When an indicator composition of the present invention is used, in various embodiments of the composition of the present invention, detection methods can be employed by filling and storing said composition in a closely-packed containers, followed by exposing said composition to atmosphere to be detected, thereby detecting concentration of detection material to be detecteds such as formaldehyde gas by color change degrees.

In addition, as indicators pertaining to such detection methods, for example, as already mentioned, indicators provided with closely-packed containers in which said composition of the present invention is filled and stored and in which said closely-packed containers are any of films, tubes, stamps, and writing instruments. By composing said composition of the present invention as an ink composition, and by containing this in a stamp or an ink containment portion of a writing instrument, stamping or writing on a portion under detection atmosphere is made, thereby capable of detecting a detection material to be detected in said portion. In addition, the present composition can also be filled in packaging bags or sealed plastics not only in closely-packed tube containers. For information, closely-packing means are not limited. Further, unpacking means for opening closely-packed materials and the like are not limited, either.

When such an indicator composition of the present invention relating to such various embodiments is used, since it is aqueous gel, unlike solid indicator, color reaction easily occurs and detection result is shown very quickly. In said indicator of a dried printed material using solid indicator reagent as already mentioned, due to gas-solid reaction, it takes 8 to 24 hours for the completion of the change of colorings, however, according to the composition of the present invention, although it depends on concentration of a detection material to be detected such as formaldehyde gas and on concentration of coloring matters for coloring, for example, detection can be made in a short time for example, in about 5 to 180 minutes. In addition, since the indicator composition of the present invention is aqueous gel with fluidity, unlike indicator with a solution layer, without selecting the place for detection, detection can be made by directly applying to each kind of atmosphere including in rooms. In particular, it is preferable to employ said gel indicator composition including organic acid having sulfo group and/or metal salts thereof, since it can detect formaldehyde with stable sensitivity.

(Production Method)

The production method of the present invention is not limited and for example, an indicator composition can be obtained by adding aqueous solution of a gelling agent such as succinoglycan and the like to a color reagent of a detection material to be detected followed by stirring. In addition, in the case of an indicator composition in which a detection material to be detected is formaldehyde, the following production method is preferable.

That is, pararosaniline hydrochloride salt and water are mixed and stirred, followed by adding a sulfite reagent such as sodium disulfite, and the like thereafter stirring, and further followed by adding organic acid having sulfo group such as sulfosalicylic acid, and the like, thereafter stirring, and filtering, thereby preparing improved Schiff's reagent. Aqueous solution of a gelling agent is added to this improved Schiff's reagent, thereby obtaining an indicator composition of one embodiment of the present invention in which pH is not greater than 3 and preferably not greater than 1.5.

The description of this application claims benefit of priority based on Japan Patent Application No.2003-142520 the entire same contents of which are incorporated by reference herein.

### EXAMPLES

### Examples 1 to 9 and Comparative Example 1

By the composition shown in Table 1, purple colored or red colored aqueous solution of pararosaniline hydrochloride salt was obtained by mixing pararosaniline hydrochloride salt and ion exchange water, followed by stirring for 1 hour. Next, to this aqueous solution, sulfite reagent such as sodium disulfite and the like were added and were stirred for 30 minutes, followed by breaching this purple or red color. Subsequently, organic acid having sulfo group such as sulfosalicylic acid and the like was added and was stirred for 30 minutes, followed by adding activated carbon and was left for 1 hour to make it transparent liquid, followed by filtering with filter paper No. 5C, thereby preparing improved Schiff's reagent. To this improved Schiff's reagent, aqueous solution of a gelling agent such as succinoglycan, agar, and the like was added, thereby obtaining a colorless to a white transparent gel indicator composition.

TABLE 1
(wt %)

| | | | | | | | | | | | Comparative |
|---|---|---|---|---|---|---|---|---|---|---|---|
| | | Examples | | | | | | | | | Examples |
| Composition | | 1 | 2 | 3 | 4 | 5 | 6 | 7 | 8 | 9 | 1 |
| Pararosaniline | | 0.05 | 0.05 | 0.05 | 0.05 | 0.20 | 0.20 | 0.10 | 0.05 | 0.05 | 0.05 |
| hydrochloride salt | | | | | | | | | | | |
| Sodium disulfite | | 0.10 | 0.10 | 0.10 | 0.10 | 0.40 | 0.20 | 0.20 | 0.10 | 0.10 | 0.10 |
| Sulfosalicyclic | | 1.00 | | 0.20 | 0.20 | 1.00 | | | | 1.00 | |
| acid | | | | | | | | | | | |
| p-toluene sulfonic | | | 1.00 | | | | 1.00 | 0.50 | | | |
| acid | | | | | | | | | | | |
| Hydrechloride | | | | 0.20 | | | | | 0.60 | | |
| Citric acid | | | | | | 0.60 | | | | | |
| Succinoglycen | | 0.80 | 0.80 | | | | | | 0.80 | | 0.80 |
| Alceseagum ® | | | | | 1.60 | 1.60 | | | | | |
| Welan gum | | | | 0.80 | | | 0.80. | | | | |
| Guar gum | | | | | | | | 1.60 | | | |
| Ager | | | | | | | | | | 0.80 | |
| Water | | 98.05 | 98.05 | 94.65 | 94.08 | 92.80 | 93.80 | 93.10 | 94.45 | 98.05 | 98.95 |
| Glycerin | | | | 4.00 | 4.00 | 4.00 | 4.00 | | 4.00 | | |
| Propylene glycol | | | | | | | | 4.00 | | | |
| Total | | 100.00 | 100.00 | 100.00 | 100.00 | 100.00 | 100.00 | 100.00 | 100.00 | | 100.00 |
| pH ofindicator | | 1.3 | 1.2 | 1.1 | 2.5 | 1.4 | 1.3 | 1.4 | 1.1 | | 3.5 |
| composition | | | | | | | | | | | |
| Viscosity | | 7,680 | 7,840 | 9,020 | 14,280 | 14,470 | 11,250 | 12,450 | 8,930 | | |
| (mPa7 · s) | | | | | | | | | | | |
| Jelly strength | | | | | | | | | 120 | | |
| (g/cm²) | | | | | | | | | | | |
| Gas | Color | Purple | Purple | Purple | Purple | Purple | Purple | Purple | Purple | Purple | Initially |
| | change | | | | | | | | | | colored |
| | after | | | | | | | | | | |
| | 0.5 ppm | | | | | | | | | | |
| | exposure | | | | | | | | | | |
| | Color | Pink | Pink | Pink | Pink | Pink | Pink | Pink | Pink | Pink | |
| | change | | | | | | | | | | |
| | after | | | | | | | | | | |
| | 0.1 ppm | | | | | | | | | | |
| | exposure | | | | | | | | | | |
| | Color | Light | Light | Light | Light | Light | Light | Light | Light | Light | |
| | change | pink | pink | pink | pink | pink | pink | pink | pink | pink | |
| | after | | | | | | | | | | |
| | 0.005 ppm | | | | | | | | | | |
| | exposure | | | | | | | | | | |
| | Color | No color | No color | No color | No color | No color | No color | No color | Light | No color | |
| | change | change | change | change | change | change | change | change | pink | change | |
| | after | | | | | | | | | | |
| | 0.001 ppm | | | | | | | | | | |
| | exposure | | | | | | | | | | |

| | | | | | | | | | |
|---|---|---|---|---|---|---|---|---|---|
| HCHO in water: | Purple | Purple | Purple | Purple | Purple | Purple | Purple | Purple | Purple |
| 0.5 mg/L | | | | | | | | | |
| HCHO in water: | Purple | Purple | Purple | Purple | Purple | Purple | Purple | Pink | |
| 0.25 mg/L | | | | | | | | | |

(Evaluation)

Next, evaluation samples were prepared by filling each composition in Examples and Comparative Examples shown in Table 1 in polyethylene containers of 5 ml by 3 g each, thereby evaluating color-change characteristics on formaldehyde gas and aqueous solution of formaldehyde.

(Color-change Tests of Formaldehyde Gas)

Evaluation samples of each of the above mentioned indicator compositions were placed in a desiccator, followed by sealing gas whose formaldehyde concentration is known, thereby conducting exposure tests.

(Color-change Tests of Aqueous Solution of Formaldehyde)

Presence of color-change was confirmed by adding aqueous solution of formalin whose concentration is known to each of the above mentioned evaluation samples. The results are shown in Table 1. From Table 1, it is recognized that all the compositions of Examples have good detection characteristics.

In addition, since the indicator composition of the present invention comprises an aqueous gel composition comprising at least a color reagent of a detection material to be detected such as formaldehyde gas and the like, water, and a gelling agent, it is easy to handle and moreover, concentration of a detection material to be detected such as formaldehyde gas and the like in a certain atmosphere can be detected in a short time.

### Examples 10 to 13

(Ozone)

An indicator composition related to Examples 1 and 12 for detecting ozone by adding dyes shown in Table 2 to and dissolving in aqueous solution of gelling agents in which glycerin, gelling agents, and water are compounded.

(Sulfur Oxide)

By the composition shown in Table 2, pararosaniline is dissolved in water, and is further dissolved by adding acids. Then, formalin solution is added. Transparent solution is obtained by purifying with activated carbon. After that, this solution and a gelling agent solution in which glycerin, gelling agents, and water are compounded are mixed and stirred, thereby obtaining an indicator composition related to Example 10 for detecting sulfur oxide.

(Nitrogen Oxide)

By the composition shown in Table 2, anthraquinone dye are added to and dissolved in a gelling agent solution in which glycerin, a gelling agent, and water are compounded. Salzmann's reagent and said gelling agent solution are mixed and stirred, thereby obtaining indicator compositions related to Examples 12 and 13 for detecting nitrogen oxide. In addition, Salzmann's reagent was adjusted by mixing 15 ml of phosphoric acid and 200 ml of distilled water and by dissolving 0.5 g of sulfanilic acid and 25 mg of N-1-naphtyl ethylene diamine dihydrochloride salt, followed by further adding distilled water to prepare 500 ml in amount.

TABLE 2
(wt %)

| | Examples | | | |
|---|---|---|---|---|
| Composition | 10 | 11 | 12 | 13 |
| Pararosaniline | 0.05 | | | |
| hydrochloride salt | | | | |
| C. I. Acid Red 9 | | 0.05 | | |
| C. I. Acid Blue 45 | | | 0.05 | |
| Salzmann's reagent | | | | 25.00 |
| Succinoglycan | 0.08 | 0.10 | 0.10 | 0.8 |
| Hydrochloride | 0.60 | | | |
| Formalin | 0.10 | | | |
| Water | 95.17 | 98.85 | 95.85 | 70.93 |
| Glycerin | 4.00 | 4.00 | 4.00 | 4.00 |
| Total | 100.00 | 100.00 | 100.00 | 100.00 |
| Color change test | — | Red to | Blue to | — |
| (ozone) | | colorless | colorless | |
| Color change test | — | — | Blue to | Colorless |
| (nitrogen oxide) | | | colorless | to pink |
| Color change test | Pink | — | — | — |
| (sulfur oxide) | to | | | |
| | purple | | | |

Note)
C. I. Acid Red 9: azo dye
C. I. Acid Blue 45: anthraquinone dye

(Evaluation)

Next, color-change characteristics were evaluated by preparing evaluation samples in which 1 g of each of the compositions of Examples shown in Table 2 is filled in 5 ml polyethylene containers.

In the case of ozone, by using an ozone generator, the above mentioned samples were exposed under the condition where CT value is 1,000 ppm·min. Color change in said samples after exposure was confirmed by visual observation.

In the case of sulfur oxide, 3 ml of 5% aqueous solution of sulfite was added to 31 of a closely-packed container, thereby leaving said samples for 1 hour. Color change in said samples after exposure was confirmed by visual observation.

In the case of nitrogen oxide, said samples were left exposed for 30 minutes to the atmosphere which includes nitrogen oxide gas generated according to a method for testing color fastness JISL0855. Color change in said samples after exposure was confirmed by visual observation.

As shown in Table 2, indicator compositions of each Example change colors change colors when contacting with atmosphere of each detection material to be detected, thereby capable of detecting preferably.

Industrial Applicability

The composition of the present invention can be utilized as an indicator capable of easily detecting concentration of a detection material to be detected in a short time such as formaldehyde, ozone, sulfur oxide SOx, nitrogen oxide NOx, and the like under a certain atmosphere.

Therefore, when the indicator composition of the present invention is included in for example, films and tubes, or when the indicator composition of the present invention is included in stamps or writing instruments as ink compositions, when in use, when films are excavated, or squeezed out of tubes, thereafter stamping, writing, or the like, said composition can be exposed to said atmosphere, thereby capable of detecting concentration of a detection material to be detected such as formaldehyde, and the like, immediately.

What is claimed:.

1. An indicator composition comprising a gel composition which comprises a color reagent of a detection material to be detected.

2. The indicator composition as set forth in claim 1, wherein said detection material to be detected is any material of formaldehyde, ozone, sulfur oxide, and nitrogen oxide.

3. The indicator composition as set forth in claim 1, wherein not only the detection material to be detected included in gas is detected but also the detection material to be detected included in liquid is detected.

4. The indicator composition as set forth in claim 1, wherein said indicator composition comprises the gel composition having a thixotropic property and comprising the color reagent of the detection material to be detected.

5. An indicator composition comprising at least a coloring reagent detecting a detection material to be detected, water, and a gelling agent.

6. The indicator composition as set forth in claim 5, wherein said gelling agent is the thixotropic agent.

7. The indicator composition as set forth in claim 6, wherein said gelling agent is succinoglycan.

8. The indicator composition as set forth in claim 5, wherein said gelling agent is agar.

9. The indicator composition as set forth in claim 1, wherein viscosity of said gelling composition measured by an E type viscometer (E viscometer) at a temperature of 20° C. is not less than 1,000 mPa·s.

10. The indicator composition as set forth in claim 1, wherein gelly strength of said gelling composition measured according to a measuring method (NIKKANSUISHIKI SOKUTEIHOU) is 30 to 2000 g/cm².

11. The indicator composition as set forth in claim 5, wherein said color reagent is contained in 0.005 to 5.0% by weight in coloration component amount with respect to the total amount of the composition of the present invention.

12. The indicator composition as set forth in claim 5, wherein said gelling agent is contained in 0.1 to 5.0% by weight with respect to the total amount of the composition of the present invention.

13. The indicator composition as set forth in claim 5, wherein following components are contained with respect to the total amount of the indicator composition;
a) 0.005 to 5.0% by weight of the color reagent in coloration component amount,
b) 0.1 to 5.0% by weight of the gelling agent, and
c) not less than 50% by weight of water.

14. The indicator composition as set forth in claim 1, wherein said color reagent is a formaldehyde color reagent.

15. The indicator composition as set forth in claim 14, wherein said color reagent comprises at least one or more color reagents selected from a group of a pararosaniline derivative, 4-amino-3-hydrazino-5-mercapto-1,2,4-triazole, and Congo red.

16. The indicator composition for detecting formaldehyde, wherein said indicator composition comprises the gelling composition which comprises Schiff's reagent, an acidity substance, and water.

17. The indicator composition as set forth in claim 16, wherein said Schiff's reagent is the reagent including pararosaniline derivative as a coloration component and including sulfurous acid or salts thereof as a decoloring reagent, and said pararosaniline derivative is hydrochloride salt of pararosaniline or hydrochloride salt of rosaniline and are included in said gel composition in a state of being dispersed in water, and said para rosaniline derivative is decolored by said sulfurous acid or salt thereof and is colored under the atmosphere of formaldehyde.

18. The indicator composition as set forth in claim 16, wherein said acidity substance includes organic acids having sulfo group.

19. The indicator composition as set forth in claim 18, wherein said organic acids are one or more organic acids having sulfone group selected from the group of sulfosalicylic acid, p-toluenesulfonate, sulfamic acid, and metal salts thereof.

20. The indicator composition as set forth in claim 16, wherein hydrogen ion concentration is not greater than pH 3.

21. An indicator composition comprising a gel composition which comprises a color reagent of formaldehyde, wherein the above mentioned color reagent is the color reagent which is colored by atmosphere of formaldehyde of not less than 0.05 ppm and by liquid atmosphere whose concentration is not less than 0.25 mg/l.

22. The indicator composition as set forth in claim 16, wherein said pararosaniline derivative is contained in 0.005 to 2.0% by weight and sulfurous acid or salt thereof is contained in 0.05 to 5% by weight with respect to the total amount of said indicator composition.

23. An indicator ink comprising a gel composition, a color reagent, a water-soluble organic solvent, and water.

24. The indicator ink as set forth in claim 23, wherein the following components are contained with respect to the total amount of the ink;
a) 0.005 to 5.0% by weight of the color reagents in a coloration component,
b) 0.1 to 5.0% by weight of the gelling agent,
c) 0.1 to 20% by weight of the water-soluble organic solvent, and
d) not less than 50% by weight of water.

25. An indicator ink containing a gel composition and comprising a Schiff's reagent in which pararosaniline derivative is a coloration component, an acidity substance, a water-soluble organic solvent, and water, and comprising an organic acid having a sulfo group as said acidity substance and having hydrogen ion concentration of not greater than pH 3.

26. The indicator composition as set forth in claim 1, wherein said color reagent is a sulfur oxide color reagent.

27. The indicator composition for detecting sulfur oxide, wherein said composition comprises a gel composition which comprises pararosaniline derivative and formaldehyde as a coloration component, and water.

28. The indicator composition as set forth in claim 27, wherein said pararosaniline derivative is contained in 0.005 to 2.0% by weight and said formaldehyde is contained in 0.001 to 1.0% by weight.

29. The indicator composition as set forth in claim 1, wherein said color reagent is the nitrogen oxide color reagent.

30. An indicator composition for detecting nitrogen oxide, wherein said composition is a gelling agent and comprises anthraquinone dye and Salzmann's reagent as a coloration component.

31. The indicator composition as set forth in claim 30, wherein said anthraquinone dye is contained in 0.005 to 2.0% by weight, said Salzmann's reagent is contained in 0.1 to 90% by weight with respect to the total amount of the gel composition.

32. The indicator composition as set forth in claim 1, wherein said color reagent is an ozone color reagent.

33. An indicator composition for detecting ozone, wherein said composition comprises a gel composition which comprises a coloring matter which changes colors by ozone and water, and said coloring matter is a dye selected from the group of an anthraquinone dye, an azo dye, a methine dye, a diphenyl methane dye, a triphenyl methane dye, and an indigo dye.

34. The indicator composition as set forth in claim 33, wherein said dye is contained in 0.005 to 2.0% by weight with respect to the total amount of said indicator composition.

35. A detecting method for detecting a detection material to be detected by color changes of said gel composition by exposing a gel composition comprising a color reagent of a detection material to be detected to atmosphere of said detection material to be detected.

36. The detecting method for formaldehyde as set forth in claim 35, wherein said detection material to be detected is formaldehyde and said gel composition is exposed to the atmosphere of formaldehyde of not less than 0.005 ppm.

37. The detecting method of formaldehyde as set forth in claim 36 being exposed to liquid atmosphere which includes formaldehyde whose concentration is not less than 0.25 mg/l.

38. The detection method for nitrogen oxide as set forth in claim 35, wherein said detection material to be detected is nitrogen oxide and as said color reagent, anthraquinone dye, and Salzmann's reagent which changes colors reacting with nitrogen oxide is included, and said gel composition is exposed to atmosphere which includes nitrogen oxide of not less than 0.01 ppm.

39. The detection method for sulfur oxide as set forth in claim 35, wherein said detection material to be detected is sulfur oxide and as said color reagents, pararosaniline and formaldehyde are included, and said gel composition is exposed to atmosphere which includes nitrogen oxide of not less than 0.005 ppm.

40. The detection method for ozone as set forth in claim 35, wherein said detection material to be detected is ozone and a color reagent selected from the group of anthraquinone dye, azo dye, methane dye, diphenyl methane dye, triphenyl ethane dye and indigo dye is included, and said gel composition is exposed to atmosphere which includes ozone of not less than 0.001 ppm.

41. An indicator provided with a closely-packing contained in which a gel composition comprising a color reagent of detecting materials is filled and stored.

42. The indicator as set forth in claim 41, wherein said Closely-packing container is selected from the group of a film, a tube, a stamp, and a writing instrument.

43. The indicator as set forth in claim 41, wherein said detection material to be detected is formaldehyde.

44. The indicator as set forth in claim 41, wherein said detection material to be detected is ozone.

45. The indicator composition as set forth in claim 41, wherein said detection material to be detected is sulfur oxide.

46. The indicator composition as set forth in claim 41, wherein said detection material to be detected is nitrogen oxide.
